(19) United States
(12) Patent Application Publication
(10) Pub. No.: US 2023/0173147 A1
(43) Pub. Date: Jun. 8, 2023

(54) BLOOD SEPARATION SYSTEM AND BLOOD PRODUCTS

(71) Applicant: Hemoclear BV, Zwolle (NL)

(72) Inventor: Arno Pieter Nierich, Hattem (NL)

(73) Assignee: Hemoclear BV, Zwolle (NL)

(21) Appl. No.: 17/906,648

(22) PCT Filed: Mar. 22, 2021
(86) PCT No.: PCT/NL2021/050190

§ 371 Date: Sep. 19, 2022

(30) Foreign Application Priority Data
Mar. 20, 2020 (NL) 2025177

(51) Int. Cl. A61M 1/02; A61M 1/36

(52) U.S. Cl. CPC A61M 1/0281; A61M 1/3633

(57) ABSTRACT

A blood filtering apparatus for recovering blood components from blood comprises an inlet for the blood; a cell filter configured for filtering a portion of the blood and retaining as a retentate a fraction of the blood containing red blood cells and platelets and passing as a filtrate a fraction of the blood, in particular mainly as plasma, containing platelets and being depleted of red blood cells. The cell filter has a pore size in a range of 2,0-3.0 micron. Further, a method and a blood product are provided.

### TECHNICAL FIELD

The present disclosure relates to preparation of blood products. In particular, the present disclosure pertains to a blood filtering apparatus and a method for the recuperation of blood components from blood, in particular for an autologous blood transfusion and a system therefor.

### BACKGROUND

It is well known that humans may benefit from receiving blood and/or blood products in cases like diseases, wounds, treatment and/or surgery. This also holds for other mammals, in particular domesticated and/or captive animals such as pets, work- and/or competition animals (e.g. dogs, horses, camels, etc. farm animals, laboratory animals) and show and/or breeding animals (e.g. zoo and/or circus animals).

In the following, for practical purposes, this disclosure focuses on (use for) humans, but unless otherwise specified it should be considered equally applicable to mammals as set out above.

Blood comprises red blood cells (“RBCs” or erythrocytes), white blood cells (“WBCs” or leukocytes) and platelets (thrombocytes) in blood plasma.

Autologous blood transfusion, the reinfusion of a patients’ own RBC containing blood component, minimises risks linked to blood transfusions with blood donated by other people, so-called homologous blood transfusions, viz. anaphylactic reactions and/or donor-associated infections such as hepatitis, acquired immune deficiency syndrome (AIDS), adverse effects HLA (human leucocyte antigens), and malaria.

In an autologous blood transfusion as well as in a homologous blood transfusion, blood to be (re)infused into the recipient may be desired to be as rich as possible in healthy red blood cells and possibly also in platelets. These RBCs and/or platelets must be collected from collected blood and possibly freed or washed from any undesired particles, which may be considered impurities and/or contaminants before (re)infusion. Although whole blood directly collected from a (dedicated) puncture may have little to no impurities and/or contaminants, some impurities in whole blood may be, among others, activated coagulation factors, antibodies to ABO incompatible acceptors, plasma free haemoglobin, leucocytes and lipids. It is noted that whole blood directly collected from a (dedicated) puncture differs from shed blood and wound drained blood by the absence, in general, of impurities like bone and tissue fragments, blood clots and fat particles, activated coagulation factors, and denaturated proteins.

Generally, for purification or separation of components from blood, in particular for purification or separation of autologous blood, the blood is put in a centrifuge chamber for separation of the relatively heavy blood cells, such as RBC’s and leucocytes, from the blood plasma and relatively light and small particles such as platelets, plasma proteins and antibodies. In some cases, the blood is also first “washed”, by mixing the blood with a “washing fluid”, usually a saline solution or Ringer’s solution. Possibly together or before such mixing, the blood may be filtered relatively coarsely to remove accidental large particulate matter and/or emboli. Such cell (washing and) separation technique works only for batches. Further, during separation by the centrifuge-process, part of the collected red blood cells become damaged and are lost to the patient. The centrifuging method also depends on complex and expensive equipment and dedicated skilled personnel to perform the centrifugal blood collection and separation technique.

Further steps are required in case blood plasma and/or plasma products are desired for reinfusion and/or for transfusion.

It is noted that filtering of whole blood or blood components is known; e.g. EP 0 518 975 discloses an apparatus for recycling autologous blood from a patient for reinfusion back to the patient comprising suction means, admixing means for admixing aspirated blood with a washing fluid, filtering means for filtering the admixture through an emboli filter and a membrane filter, monitoring means for measuring the amount of cellular component volume in the filtered blood, filtration means for removing excess fluid and particulates from the blood, and reinfusion means. The membrane filter may be any conventional membrane-type separator with a pore-size ranging from 40000 Daltons to 400000 Daltons molecular weight cut-off. However, if larger impurities are to be removed, a plasma filter having a pore size larger than about 400000 Daltons, and up to 0.4 µm, can be used.

WO 93/01858 discloses a similar system with a maximum pore size of the platelet filter of 0.6 µm.

WO 2008/028975 discloses a blood filtering device for the recuperation of blood from wound drained blood having a first filter arranged upstream of a platelet filter. The first filter is adapted for removing emboli and/or large particulate matter from the blood and for allowing red blood cells to pass. The platelet filter is adapted for retaining red blood cells. An exit port is arranged downstream of the first filter and upstream of the platelet filter. In the device the platelet filter has a pore size of more than about 0.5 µm. A method of recuperating blood from wound drained blood is also disclosed.

US 7,794,420 discloses a process for treating the hemorrhage fluids of a patient at the time of a surgical procedure with the aim of carrying out an autotransfusion. The process includes a stage for recovering the hemorrhage fluids with simultaneous introduction of agents such as anticoagulant and/or diluting agents, at least one stage for the mechanical separation/concentration of this hemodiluted hemorrhage fluid in order to concentrate its content in red blood cells and partially purify it, this phase being collected in a sterile manner so as to be fit for injecting back to the patient. Also described is the related device.

US 4,631,050 discloses an autotransfusion system for salvaging, washing, and concentrating blood and returning the blood to a patient during surgery. The system comprises both a filtration unit and an ultrafiltration unit. The ultrafiltration unit is configured so as to communicate with the filtration unit, and both the filtration unit and the ultrafiltration unit are provided with a suitable conduit for conveying blood back to a patient. The ultrafiltration unit is divided into two chambers by a semipermeable membrane, the membrane being selected so as to permit fluid to pass therethrough, while preventing the passage of blood cells and other formed elements.

EP 0 070 738 A discloses a process and apparatus for plasmapheresis, which process comprises conduction of blood over a microporous membrane in reciprocatory pulsatile flow.

US 2005/0133447 relates to a process and apparatus for separating blood plasma, having a mixing unit in the form of a first injection having a first connecting tube and a first piston to provide a compartment for a mixture composed of plasma to be separated and the protein-precipitating agent; a separating unit composed of a filtering tube for separating and preserving a solid material after separation including further tubes and pistons.

Further, US 10 065 134 relates to an integrated leukocyte, oxygen and/or CO₂ depletion, and plasma separation filter; US 6 099 730 relates to an apparatus for treating whole blood comprising concentric cylinders defining an annulus therebetween; US 7 182 865 relates to a device for separating whole blood under gravitational force; and US 4,871,462 relates to enhanced separation of blood components.

In view of the above, improvements are desired.

### SUMMARY

Herewith is provided A blood filtering apparatus for recovering blood components from blood, the apparatus comprising
an inlet for the blood;
a cell filter configured for filtering a portion of the blood and retaining as a retentate a fraction of the blood containing red blood cells and platelets and passing as a filtrate a fraction of the blood containing platelets and being depleted of red blood cells. The cell filter has a pore size in a range of 2.0-3.0 micron.

The apparatus enables filtering red blood cells from the blood and separating the blood in a red blood cell rich fraction in the retentate and a red blood cell poor fraction in the filtrate. Since mammalian, in particular human, red blood cells tend to have an average size of about 4 micron, a filtering apparatus according to the present disclosure enables retaining healthy red blood and letting fluid and smaller impurities and/or contaminants pass. Further, it is believed that, without wishing to be bound to any specific theory, red blood cells may tend to flex and to pass through smaller openings than the diameter of a red blood cell at rest.

Since for blood transfusion the most relevant component of the blood to be transfused is (are) the red blood cells, red blood cell rich blood (blood having a high haematocrit) is particularly desired for transfusion and can be readily produced using the present apparatus.

It has been found that a pore size in the range 2.0-3.0 micron is very effective in retaining red blood cells. Moreover, it has been found that such a pore size is also very effective in retaining a portion of the platelets in the blood to be filtered; such pore sizes have proven to enable retaining significant fractions of the platelets of human blood. At the same time, such pore sizes have proven to let other blood components pass without the filter getting clogged. Thus, operation of the apparatus may be reliable. E.g., large proteins, already haemolyzed cellular RBC parts, or large cellular tissue fragments will pass through the membrane entering into the filtrate and will not contaminate the retentate.

The retentate of filtering whole blood or fresh lost blood with such apparatus will contain both most important cellular components in tissue oxygenation and haemostasis.

Presently, platelet rich and red blood cell rich compositions are manufactured by blood product providers like blood banks from red blood cell concentrates and platelet concentrates, each being produced by centrifuging as set out above with associated costs and low efficiency. Further, manufacture of platelet concentrates requires further processing steps further increasing effort and cost and the platelet concentrates tend to have very limited shelf life, in particular compared to blood and “packed cells” as provided by blood banks. Therefore, in present blood transfusions, such platelet rich and red blood cell rich compositions are manufactured shortly before transfusion of the composition from red blood cell concentrates and platelet concentrates of different donors.

The presently provided apparatus facilitates provision of a platelet rich and red blood cell rich composition. Moreover, the respective components of the composition may inherently originate from a single individual, reducing risks from combining components from different individuals into one composition for complications in transfusions (autotransfusion or homotransfusion).

The pore size of the cell filter further provides a relatively low fluid flow resistance, so that the device may operate with low or no applied pressure difference across the filter. Thus a relatively simple device is achieved which allows recuperating red blood cells and platelets with minimum additional equipment being necessary and/or without moving parts in the apparatus which might cause shear stress on the RBCs and/or platelets.

E.g., a fluid pressure difference across the filter may be provided of about 0.1 bar or less overpressure on the upstream side and/or about 0.1 bar or less underpressure on the downstream side (0.1 bar equals 1 m water column or 100 kPa). At such pressure differences, RBCs and platelets tend to remain undamaged. Such fluid pressures may be provided by arranging a source of blood to be filtered, e.g. a pouch, about 1 meter above the cell filter of the apparatus, and arranging a receptacle for the filtrate and/or the retentate, e.g. a pouch or set of pouches, about 1 meter below the cell filter of the apparatus and fluidly connecting the source across the filter and/or a housing comprising the filter, preferably such that a substantially continuous column of fluid is provided between the source and receptacle e.g. using appropriately sized tubing. Such arrangement may be readily provided using a standard infusion pole.

In case of filtering fresh drawn blood from a dedicated puncture, RBCs tend to be intact and the filtrate may be a rather pure and RBC-poor or RBC-free plasma containing a fraction of the original platelets, those that were able to pass through the filter.

Herein, unless otherwise specified or clear from the context, “blood” may generally refer to any variety of liquid RBC-containing blood such as circulating whole blood, fresh lost blood such as whole blood drawn from a puncture, post trauma/surgery lost blood over a prolonged time called shed blood, and RBC-containing blood products for such as stored blood for blood transfusion.

When blood is filtered in the apparatus, e.g. whole blood, the blood may be provided with an anticoagulant like heparin or Citrate phosphate dextrose (CPD). Also or alternatively an additive solution (AS) for the RBCs such as SAGM (saline, adenine, glucose, mannitol) may be provided as storage solution for the RBCs. Also or alternatively, the retentate may be provided with an anticoagulant or storage solution such as heparin, CPD or SAGM.

Thus, autologous blood can be separated into a mostly cellular part and a more non cellular part. The separation described into this disclosure comprises portions specifically related to the separation of blood into a component that is rich in RBCs and into a component that is poor in RBCs or, preferably, substantially devoid of RBCs. As well as portions specifically related to the separation of blood into a component that is rich in platelets and into a component that is poor in platelets. In addition, plasma containing useful antidotes, such as antibodies, may be obtained.

Note that a red blood cell-rich blood may be (re)infused into the recipient under low pressure through a leukocyte filter for further reducing numbers of possibly remaining white blood cells in the transfused blood.

In the apparatus, the pore size of the cell filter may be in a range of 2.1-2.7 micron, in particular in a range of 2.2-2.7 micron, more in particular in a range of 2.2-2.5 micron, such as in a range of 2.3-2.4 micron.

It has been found that such pore size is not only very effective in retaining red blood cells but that it also tends to retain a significant portion of platelets in the blood.

A pore size in a range of 2.2-2.5 micron has been found to enable retaining on the order of one half of the number platelets in the blood and to let the other half of the platelets pass through the filter. A pore size in a range of about 2.3-2.4 micron tends to provide best results; over 40% of the platelets are reliably retained. Thus, the retentate of the cell filter provides a platelet rich and red blood cell rich composition. Conversely, the filtrate of the cell filter provides a platelet poor and red blood cell poor composition.

Presently, platelet rich and red blood cell rich compositions may be manufactured from red blood cell concentrates and platelet concentrates, each being produced by centrifuging as set out above. Further, manufacture of platelet concentrates requires further processing steps further increasing effort and cost and the platelet concentrates tend to have very limited shelf life.

The presently provided apparatus facilitates provision of a platelet rich and red blood cell rich composition. Moreover, the respective components of the composition may inherently originate from a single individual, reducing risks from combining components from different individuals into one composition.

The apparatus may comprise a platelet filter for filtering a portion of the filtrate of the cell filter into a platelet rich fraction and a platelet depleted fraction.

The filtrate of the cell filter comprises platelets. It has been found that the presently provided apparatus facilitates provision of such different plasma products which would otherwise be complicated and expensive to make.

The platelet filter may be a standard haemoconcentration device, e.g. based on ultrafiltration techniques. The platelet depleted fraction is mainly water and may be considered waste.

Also or alternatively, the apparatus may comprise a second filter for filtering a portion of the filtrate of the cell filter, which second filter may have a pore size in a range of 0.2-0.6 micron, in particular in a range of 0.3-0.5 micron, preferably in a range of about 0.3-0.4 micron. This may serve for fresh plasma apheresis, and a concentrated waste fraction may be obtained which may comprise (fragments of) RBCs and platelets and/or other substances considered waste to the plasma; however coagulation factors and/or antibodies may remain in the filtrate. The latter may be employed for so-called passive vaccination without any cellular components. At least part of the waste fraction, also referred to as cellular waste, may be further refined in another process to salvage possibly useful products contained therein.

Thus, the filtrate of the cell filter may provide a so-called platelet poor plasma, in particular in case the filtered blood is fresh drawn whole blood. The filtrate may be transformed to a platelet rich plasma by extracting liquid, in particular water, from it. Platelet rich plasma may be used as a therapeutic agent for cases including wound healing, hemostatic therapy, tissue regenerative solution, cosmetic treatments and burn treatment. The presently provided apparatus facilitates provision of platelet poor plasma and of concentrating that into platelet rich plasma by the platelet filter.

The apparatus may be employed during or after a surgical procedure for filtering blood from the patient subject to the surgical procedure. E.g. blood may be withdrawn or collected and filtered using the apparatus as described, the RBC-rich retentate may be reinfused, during or after the procedure, as autologous transfusion, while the platelet rich fraction of the plasma may be used, e.g. at the end of surgery, for application in the surgical wound area for hemostatic and regenerative purposes as autologous material.

Generally, platelet rich plasma may be manufactured in batches using centrifuge processes as indicated before. The provided apparatus allows continuous or quasi continuous manufacturing of platelet rich plasma, also enabling provisions of large amounts at little effort.

The filtrate of the cell filter may contain antibodies against pathogens. Therefore at least part of the filtrate may be donated to a recipient for vaccination (prophylaxis) and/or therapeutic purposes: passive vaccination. In some cases, in particular for prophylactic purposes, donation of platelet poor plasma, e.g. the filtrate of the cell filter and/or further filtered with the optional second filter may be used.

The filter material of at least the cell filter may comprise a polymer layer, in particular a polymer surface layer, preferably the filter being a polymer membrane. The same may apply for the platelet filter.

This has been found to facilitate manufacturing the filter and to prevent damage to the red blood cells and/or to prevent damage to the platelets.

Particularly suitable materials are polyesters and/or are selected from the group consisting of polyurethane, in particular polyester polyurethane, polyethylene terephthalate (“PET”), or polyethylene furanoate (“PEF”), or poly(propylene furan-2,5-dicarboxylate) (“PPF”). Such materials have excellent chemical and physical characteristics for processing blood without negatively affecting the blood component.

Films of a biaxially-oriented polyethylene terephthalate (BoPET) polymer material such as “Mylar”, “Melinex” and “Hostaphan” have been found particularly successful.

Such films may have very smooth finish (e.g. having a typical surface structure size other than the pores of smaller than about 100 nm, preferably smaller than 50 nm such as smaller than 25 nm) and allow reliable provision of pores of the desired sized. Moreover, the materials may be processed by welding and gluing facilitating manufacture of the apparatus. Also, the materials can be sterilized well.

The membrane may be substantially free and unsupported other than at the edges, at least unsupported by any structure fixed to the membrane apart from at or near a circumference.

The filter material of at least the cell filter may be a track-etched membrane. The platelet filter may also be a track-etched membrane.

Track etched pores tend to have predictable diameters and tend to have predictable direction. This facilitates designing a filter material with a predictable porosity and smooth surface structure. Further, entrant and exit edges of the pores may be devoid of burrs etc. leaving the surface of the membrane into which the pores have been made with a smooth finish preventing damage to cells in the blood contacting the membrane, e.g. when sliding over the membrane surface. Also through holes may be reliably formed meaning few blind holes in which particles (in particular platelets) could get stuck and be lost to a process performed with the filtering apparatus.

At least the cell filter may comprise plural filter portions arranged opposite each other defining a first flow path for unfiltered blood and retentate between the opposite filter portions and defining a second flow path comprising plural second flow path portions extending through different ones of the filter portions, for collecting filtrate downstream of the filter portions.

By such multi-layer filter construction, a relatively large filter surface may be provided in a relatively compact apparatus. Also, a relatively short way through the blood to the nearest filter may be provided so that a thick layer of blood, compared to a single-sided filter setup could be used. This facilitates a relatively rapid filtering process.

At least some of the plural second flow path portions may be in fluid communication with each other at least downstream of the cell filter. This may facilitate construction of the filter as well as improving a constant flow of the filtrate.

At least some of the opposite filter portions may be spaced by a retentate spacer layer allowing flow of blood and retentate through the respective spacer layer it and/or at least some of the opposite filter portions may be spaced by a filtrate spacer layer allowing flow of filtrate through the respective spacer layer. The respective spacer layers prevent that the opposite filter portions contact each other and possibly stick to each other, hindering subsequent blood flow and/or filtrate flow. Further, compressive damage to the red blood cells and/or platelets may be prevented and/or regulation of pressures across a filter portion may be improved preventing squeezing of undesired components through the filter. Also or alternatively pressure onto, or across, the filter portions may be prevented. This is considered to improve filtration reliability and filter robustness. Moreover, a constant and even flow over and across the filter portions may be improved.

The spacer may comprise or be at least one of a fibrous material, in particular a monofilament-based material, a mesh material, a woven or knit cloth, in particular a warp woven, or warp knitted cloth.

Such spacer materials may be effectively made in large volumes or rather in large lengths of layer at comparably low cost. Further, they may combine a comparably large separation distance with a low amount of obstruction (pillars) in the layer, e.g. a loosely knit thick fabric layer. At least part of the spacer material may be a mesh cloth. Suitably, the spacer is a monofilament warped singular, double manifold loin-weaved cloth which may have a hexagonal form structure. The spacer material may be polyamide.

Preferably the spacer layer has crossing wires or filaments with a minimum thickness of 50 micron, preferably 100 micron, and flexibility allowing deformation of the wire/filament to a bending radius down to 10 or even down to 5 times the thickness, preferably being so deformable reversibly and/or elastically. Thus, the spacer layer will provide a minimum thickness when compressed of at least the combined thickness of the crossing wires while still retaining open pores larger than once a minimum thickness, ensuring a flow path between the opposite filter portions.

The cell filter and/or the platelet filter may comprise a plurality of filter portions stacked together in a stacking direction and being configured for, seen in the stacking direction, providing alternating first flow paths for retentate of the filter and second flow paths for filtrate of the filter, wherein in particular in the stack between adjacent filter portions spacing layers are provided, in particular the stack comprising a repeating sequence of a filter portion and a spacer layer.

By such filter construction, a relatively large filter surface may be provided in a relatively compact apparatus. Edges of filter portions of the stack may be suitably attached together, e.g. glued or welded, to define and separate the different flow paths and prevent leakage and/or contamination.

The apparatus may comprise an inlet for the blood to be filtered. The inlet may connect to plural filter layers, associated with plural spacing layers. Also or alternatively, the apparatus may comprise a first outlet for retentate of the cell filter and a second outlet for filtrate of the cell filter. Such outlet for the retentate and/or outlet for the filtrate may connect to plural spacing layers of the respective one of the first and second flow paths.

Thus, the separate fractions may be collected as desired and/or be administered to a recipient e.g. by (re)infusion into a receiving individual. For this, dedicated equipment may be connected with the respective outlet(s), e.g. an intravenous line. In case of provision of the platelet filter, the filtrate of the cell filter may be separately let out or at least part of the filtrate may be filtered by the platelet filter. In such case, the apparatus may comprise a third and/or a fourth outlet for filtrate of the platelet filter and/or retentate of the platelet filter, when applicable. Then, such outlet for the filtrate of the platelet filter and/or outlet for the retentate of the platelet filter may connect to plural spacing layers of the respective flow paths. In view of the above, herewith a method is provided comprising separating an amount of blood comprising red blood cells and platelets into a red blood cell rich fraction and a red blood cell poor fraction.

In the method, the red blood cell rich fraction
(1) has a haematocrit of at least 30% preferably over 40% and/or in a range of 30%-50%, and/or the red blood cell rich fraction comprises at least 60% of the red blood cells of the amount of blood, preferably at least 70% more preferably 75% or more; and
(2) has a platelet concentration of about 40-450 thousand platelets per microliter, preferably in a range of 70-400 thousand platelets per microliter and/or the red blood cell rich fraction comprises at least 25% of the platelets of the amount of blood, preferably at least 30%, more preferably at least 40% or more preferably 50% or more.

The resultant red blood cell rich fraction is also platelet rich. Such blood product is beneficial to a recipient since it contains both most important cellular components in tissue oxygenation and haemostasis in a high amount. Moreover, the platelets and RBCs are obtained from the same amount of blood, e.g. from a single individual. This enables reduction of complications for transfusion of the blood and when transfused may help recovery of the recipient, in particular in case of autotransfusion.

During or after filtration, at least the cell filter may be rinsed with a suitable liquid like saline or Ringers liquid, flushing remaining retentate to an outlet and/or flushing platelets through the respective filter into the filtrate side, reducing loss of platelets that otherwise may be stuck in the filter. Such rinsing may comprise flowing a liquid through the filter against the filtering direction, e.g. for urging and/or dislodging RBCs, platelets and/or other particles from the filter into the retentate side thus preventing clogging of the filter and/or recovering the dislodged particles into the retentate.

At least part of the red blood cell rich fraction may be further processed or not to provide a blood product for transfusion into a human recipient, wherein the blood product has a haematocrit of at least 30%, preferably over 40% and/or in a range of 30-60%, and comprises about 40-300 thousand platelets per microliter, preferably in a range of 40-250 thousand platelets per microliter, and more preferably even 40-150 thousand platelets per microliter, but still more preferably in a range of 70-200 thousand platelets per microliter, e.g. in a range of 70-150 thousand platelets per microliter, and wherein the blood product essentially consists of blood components of a single donor individual. Such blood fraction may in particular be used for reinfusion into the donor (autotransfusion) providing the beneficial effects of platelets and assist recovery.

Thus, platelet rich packed cells may be produced, and in a simple and low cost manner. Since this blood product is derived from a single donor, risks of complications for a transfusion recipient receiving the blood product may be reliably assessed and prevented. Moreover, the product may have a very low plasma concentration or -content further reducing risks of complications for a transfusion recipient receiving the blood product. E.g., the platelet rich and red blood cell rich blood product may be provided from a donor having blood type O, preferably type O Rhesus negative, which may be donated to individuals of any blood type.

Note that donation of whole blood from a blood donor may provide complications due to antigens in the blood plasma, at least requiring an exact match with the recipient.

In an aspect, a method of recovering blood components from an amount of blood, in particular separating blood into components, is provided, comprising filtering at least a portion of the amount of blood using a blood filtering apparatus as described herein and collecting at least one of the retentate and the filtrate. This method may comprise the method discussed in the preceding paragraphs.

The method allows providing blood component products effectively and at little costs.

The method may comprise providing a pressure difference across the filter of up to 0.2 bar, in particular providing a fluid pressure difference across the filter of about 0.1 bar or less overpressure on the upstream side of the filter and/or about 0.1 bar or less underpressure on the downstream side of the filter (0.1 bar equals 1 m water column or 100 kPa). This may be provided by suction, pressurizing and/or use of gravity.

### BRIEF DESCRIPTION OF THE DRAWINGS

The above-described aspects will hereafter be more explained with further details and benefits with reference to the drawings showing a number of embodiments by way of example.

FIG. 1 indicates a top view of a blood filtering apparatus;

FIG. 2 indicates a cross section view of the blood filtering apparatus of FIG. 1 along section II-II;

FIGS. 3 and 4 are electron microscopy photographs of filter materials;

FIGS. 5 and 6 are (electron) microscopy photographs of spacer material;

FIG. 7 shows is an exemplary embodiment;

FIG. 8 is a schematic representation of methods of filtering blood.

### DETAILED DESCRIPTION OF EMBODIMENTS

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualised with alphabetic suffixes.

Further, unless otherwise specified, terms like “detachable” and “removably connected” are intended to mean that respective parts may be disconnected essentially without damage or destruction of either part, e.g. excluding structures in which the parts are integral (e.g. welded or moulded as one piece), but including structures in which parts are attached by or as mated connectors, fasteners, releasable self-fastening features, etc. The verb “to facilitate” is intended to mean “to make easier and/or less complicated”, rather than “to enable”.

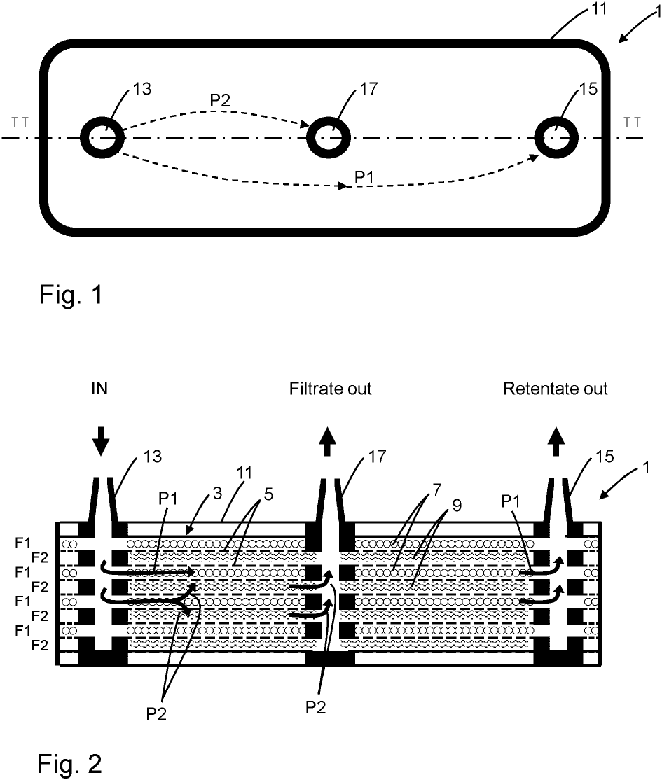

FIGS. 1 and 2 show a blood filtering apparatus comprising a filter 3 in turn comprising a plurality of filter portions 5 separated by a first set of spacer layers 7 and by a second set of spacer layers 9 stacked onto each other. The combination provides plural first filter layers F1 and second filter layers F2. The different spacer layers 7 and 9 are indicated in FIG. 2 with different hatching for clarity but their construction in the filter may be substantially identical (see below). The filter 1 may comprise more, less and/or differently sized and/or shaped filter portions. The number and/or positions of inlet(s) and/or outlets may also be chosen other than shown.

The apparatus comprises a liquid tight housing 11 around the filter 3 through which extend an inlet 13 for the blood to be filtered, an outlet 15 for retentate and an outlet 17 for filtrate. As may be seen from FIG. 1B, the inlet 13 and retentate outlet 15 are in fluid communication with the first filter layers F1 and the filtrate outlet 15 is in fluid communication with the filter layers F2.

The inlet 13 and the filter portions 5 arranged opposite each other and being separated by the first set of spacer layers 7 define a first flow path P1 for unfiltered blood and retentate between the opposite filter portions from the inlet 13 to the retentate outlet 15 through the filter layers F1. The first flow path P1 extends around the filtrate outlet 17 as may be understood from FIG. 1A.

The filter portions 5 arranged opposite each other also define a second flow path P2 comprising plural second flow path portions P2 extending through different ones of the filter portions 5, for collecting filtrate downstream of the filter portions, here at the filtrate outlet 17. The second flow path P2 extends through from the inlet 13 to the filtrate outlet 17 through both a filter layer F1 and a filter layer F2.

The inlets and/or outlets are connected to the filter portions leak tight, e.g. by welding, gluing or clamping, so that fluid communication between the filter layers F1 and F2 exists only through the pores of the filter portions so as to prevent filter bypassing of blood components from the incident side / retentate side to the filtrate side.

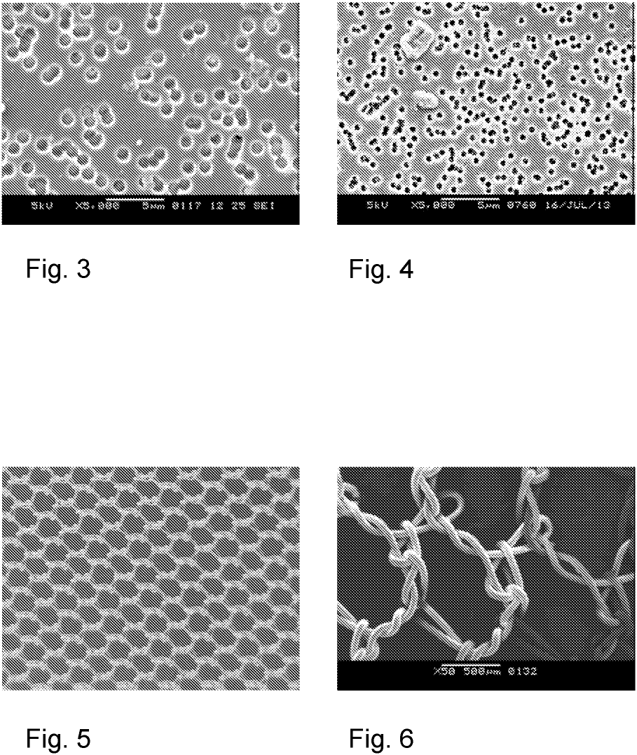

FIGS. 3 and 4 are electron microscopy photographs of track etched mylar membranes showing track etched membranes with pores of different sizes: 2.3 micron (FIG. 3) and 0.4 micron (FIG. 4) respectively. From these images, it may be seen that the pore sizes vary only to within a few percent, that the arrangement of pores is somewhat erratic and that the surface porosity (fraction open area of total surface area) may be on the order of 10-20%, preferably it is on the order of about 10-30% this may retain strength of the membrane, provide relatively smooth surface for relatively unobstructed flow of the blood cells over the surface, and still provide a relatively large open area and low flow resistance across the filter membrane.

Suitable filter membrane thickness may be in a range of 10-50 micron, preferably in a range 10-30 micron, more preferably in a range of 10-15 micron, e.g. about 12-13 micron, 15-20 micron e.g. about 17-18 micron, or 20-25 micron, e.g. about 23-24 micron. Such ranges are commercially readily available in smooth surface finish and high quality (no holes etc.). Note that the thicker the membrane is, the more robust it becomes and the better workable for manufacturing the filter, but also the higher the flow resistance across the filter membrane becomes. Different numbers of stacking, different amounts of blood to be filtered and/or different pressure differences across the filter membrane(s) in combination with filter size may determine an optimum selection.

FIGS. 5 and 6 show suitable spacer material. The shown material is a polyamide monofilament mesh sloth. The used wire is a round non-flattened smooth monofilament thread of diameter ca. 50 micron. The fabric is warped singular double-manifold loin-weaved. FIG. 5 shows that the cloth has a very open and substantially hexagonal structure. FIG. 6 shows that wire portions are repeatedly wound around each other and/or crossing each other, also forming loops. Thus, when arranged between filter membranes and compressed the spacer material will provide significant open structure so that red blood cells of average size 5 micron and/or clumps of RBCs of about 10 micron may readily pass the spacer material and the spacer material will provide, even then, little flow obstruction for the blood/retentate and even less for the filtrate as that contains smaller objects.

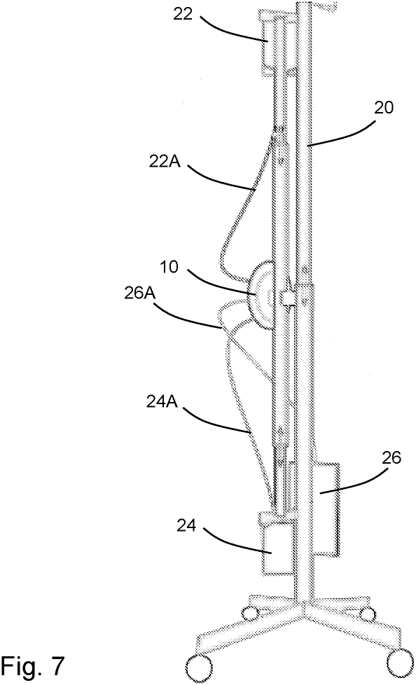

FIG. 7 shows an exemplary apparatus and a manner of operation of the apparatus. The filter 10 comprising filter 1 described hereinbefore mounted in an optional outer housing for security, is attached to a standard IV-pole 20 or another support. A blood bag 22 or other supply of blood to be filtered is attached to the IV-pole 20, above the filter 10, e.g. between about 75-150 cm above the filter 10. A retentate RBC bag 24 or other retentate container is attached to the IV-pole 20, below the filter 10, e.g. about 75-150 cm below the filter 10. A filtrate bag 26 or other filtrate container is also attached to the IV-pole 20, below the filter 10, e.g. about 75-150 cm below the filter 10. Note that instead of a common support several supports like IV-poles etc. may be used.

Referring also to FIGS. 1-2, The blood bag 22, retentate bag 24 and filtrate bag 26 (or rather: the respective containers) are connected to the inlet 13, retentate outlet 15 and filtrate outlet 17, respectively of the filter 1 with tubes 22A, 24A, 26A, respectively, or other conduits. The thus-formed assembly provides a substantially liquid tight and preferably also gas tight apparatus, preferably being sterile.

In use, the vertical arrangement of the apparatus and gravity cause blood to be filtered to flow from the blood supply 22 into the filter 1. There, the blood is flown from the inlet 13 over the filter portions 5 into and through first filter layers F1. From there, a portion of the plasma and a fraction of the platelets of the blood pass through (the pores in) the filter portions 5 into the second filter layers F2 and from there via the filtrate outlet 17 and the tubes 26A into the filtrate bag 26. Portions of the blood that have not passed a filter portion 5 form retentate are flow from the filter 1 via the retentate outlet 15 and the tubes 24A into the retentate bag 24 forming an amount of platelet rich packed cells.

Note that filtering speed and/or efficiency may be controlled by adjusting height differences between the parts 10, 22, 24, 26 in the arrangement. Also or alternatively, external pressure or suction may be applied to one or more of the bags 22, 24, 26.

After the filtering, the collected filtrate may be further filtered and/or concentrated to produce platelet rich plasma, platelet poor plasma and/or any other plasma product.

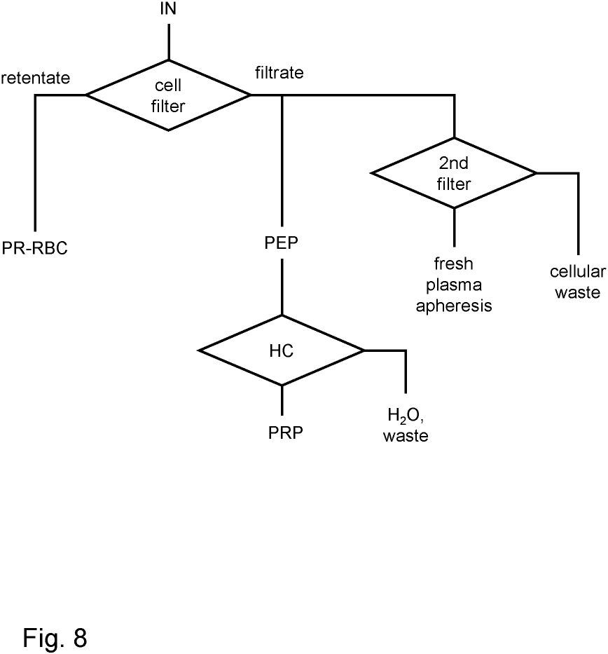

A schematic of a method is shown in FIG. 8. Blood to from which one or more blood components are to be recovered is introduced into the method at “IN”. In the filter (e.g. filter 3 in FIGS. 1-2), the blood is separated into retentate and filtrate by the cell filter. The retentate may comprise essentially all RBCs of the blood introduced into the filter. If so desired, the filter may be rinsed using a suitable fluid. A portion of the filtrate may be considered and possibly used as a platelet enriched plasma (“PEP”). Such substance is considered not achievable by other means, at least not without undue effort and costs for separating blood fractions and recombining at least part of the previously separated fractions.

The platelet enriched plasma PEP may be further processed by haemoconcentration (“HC”) into platelet rich plasma (“PRP”) (and a waste fraction being predominantly water).

Also or alternatively, a portion of the filtrate may be filtered using a second filter of about 0.4 micron to provide a fresh plasma product and particulate retentate mainly comprising platelets and cell fragments considered waste.

Uses and benefits of the various products have been explained before.

As examples, several blood filtering apparatus were provided each comprising a cell filter of the construction of FIG. 1 containing 34 membrane layers providing filter portions 5. Each membrane layer was made of 23 mm thick track etched Hostaphan PET film. The pore size of the membranes was in a range 2.2-2.6 micron, as determined by bubble-point method based on pₘₐₓ = (4 y cosθ) / d, pₘₐₓ being gas pressure at the bubble point, y surface energy of the measuring liquid, θ the wetting angle of the measurement liquid with the substrate and d the pore diameter, using nitrogen gas and as measurement liquid 30 wt% ultrapure water and 70 wt% isopropanol IPA yielding K_{theory} = 4 γ cosθ = 0.065 N/m at room temperature.

Using apparatus with a pore size determined as 2.2-2.4 micron arranged as in FIG. 7, three 500 ml units of donated whole blood were filtered and separated, by gravity-driven filtration, into a retentate rich in RBCs and platelets (“cellular component”) and a filtrate (“plasma component”). Prior to the filtering, as an option the apparatus was primed with isotonic saline. The retentate contained all of the red blood cells (“RBCs”) and white blood cells (“WBCs”) and most of the platelets. Measurement results were: retention of 100 ± 1.63% of the RBCs, 99 ± 4.5% of the WBCs and 83 ± 3.0% of the platelets of the initial whole blood; the filtrate contained no RBCs or WBCs and 12 ± 1.9% of the initial platelets. In the measurements, no significant difference was found between haemolysis prior to (0.00 ± 0.01%), and after separation (0.04 ± 0.02%, p 0.057) and platelet functionality, morphology and activation were found comparable before and after separation. Thus, the separation appeared to have little to no adverse effects on the cellular components.

Similarly, using apparatus with a determined pore size 2.2-2.4 micron arranged as in FIG. 7, six 250 ml units of donated whole blood were diluted with 0.9 % NaCl saline to 600 ml at a haematocrit of 0.20 ± 0.01% each, which diluted blood mixtures were filtered and separated, by gravity-driven filtration, into a retentate rich in RBCs and platelets (“cellular component”) and a filtrate (“plasma component”). The thus-obtained retentate was further diluted with 300 ml 0.9 % NaCl saline and the mixture was again filtered providing a second retentate and second filtrate. The second retentate was again filtered, without further liquid addition, to concentrate the second retentate into a concentrated final retentate; the filtrate of each filtering round can be collected separately or combined with filtrate of another filtering round. Thus, the initial whole blood was washed, comparable to washing of shed blood. This washing procedure yielded recovery of RBCs of at least 87 ± 6%, of WBCs of at least 93 ± 7% and of platelet of at least 68 ± 10% of the initial whole blood into the final concentrated retentate.

In each case at least part of the retentate could be returned to the donor, transfused to a receptor and/or stored, after optional addition of SAGM or another supplement and/or agent, as usual for donated blood. The white blood cells (“WBCs”) could be removed from the platelet-rich and RBC-rich cellular component using a commercial leucocyte filter before or after storage of the retentate and before transfusion of the WBC-depleted RBC-rich and platelet-rich component to a receptor. The filtrate(s) could be stored and/or transfused as well, either unprocessed, concentrated or processed otherwise. The filtrate(s) could be subject to cooling to and storage at a temperature below -10℃, preferably below -18℃ e.g. at about -25° C. for one or more days and reheated to a suitable temperature for (re-)infusion into a receptor. This may destroy and/or incapacitate most if not all remaining RBCs and/or platelets that entered into the filtrate, thus reducing possible adverse effects of the donation, even if the donor and receptor are matched for ABO- and/or Rhesus-blood type.

Similarly these apparatus were used for filtration of whole blood from a donor convalescing from a disease resulting in antigens and/or antibodies in the donor’s blood stream. As an example, whole blood from several recovering COVID-19 patients was obtained and separated as described herein (see preceding description of the whole blood separation) in a clinical setting. In each case, the retentate / cellular fraction was returned to the donor and the filtrate / plasma fraction was transfused to another patient suffering from the same disease. As a result of the filtration, the plasma fraction was substantially depleted from RBCs and had at most a small fraction of platelets, while containing most if not all of the antibodies of interest from the initial blood. It was found that the thus-obtained plasma functioned as an effective treatment, or at least treatment assistant, against the disease in diseased receptors, e.g. resulting in one or more of reduced hospitalisation duration, reduced intensive care-treatment duration and/or significantly higher probability of survival than a control group. The filtrate is also considered an effective vaccine against the disease in healthy receptors, e.g. resulting in lower hospitalisation rates.

The disclosure is not restricted to the above described embodiments which can be varied in a number of ways within the scope of the claims.

Elements and aspects discussed for or in relation with a particular embodiment may be suitably combined with elements and aspects of other embodiments, unless explicitly stated otherwise.

1. A blood filtering apparatus for recovering blood components from blood, the apparatus comprising
an inlet for the blood; and
a cell filter configured for filtering a portion of the blood and retaining as a retentate a fraction of the blood containing red blood cells and platelets and passing as a filtrate a fraction of the blood containing platelets and being depleted of red blood cells;
wherein the cell filter has a pore size in a range of 2.2-2.7 microns.

2. The blood filtering apparatus according to claim 1, wherein the pore size is in a range of 2.2-2.5 microns, .

3. The blood filtering apparatus according to claim 1,
wherein the apparatus comprises a platelet filter for filtering a portion of the filtrate of the cell filter into a platelet rich fraction and a platelet depleted fraction, and/or
wherein the apparatus comprises a second filter for filtering a portion of the filtrate of the cell filter wherein the second filter has a pore size in a range of 0.2-0.6 microns.

4. The blood filtering apparatus according to claim 1,
wherein the filter material of the cell filter comprises a polymer layer.

5. The blood filtering apparatus according to claim 1, wherein the filter material of the cell filter is a track-etched membrane.

6. The blood filtering apparatus according to claim 1, wherein the cell filter comprises plural filter portions arranged opposite each other defining a first flow path for unfiltered blood and retentate located between the opposite filter portions, and
defining a second flow path comprising plural second flow path portions extending through different ones of the filter portions, for collecting filtrate downstream of the filter portions,
wherein the plural second flow path portions are in fluid communication with each other at least downstream of the cell filter.

7. The blood filtering apparatus according to claim 6, wherein at least some of the opposite filter portions are spaced by a retentate spacer layer allowing flow of blood and retentate through the respective spacer layer and/or at least some of the opposite filter portions are spaced by a filtrate spacer layer allowing flow of filtrate through the respective spacer layer.

8. The blood filtering apparatus according to claim 7,
wherein the spacer comprises or is at least one of
a fibrous material,
a mesh material, and
a woven or knit cloth,.

9. The blood filtering apparatus according to ,
wherein the cell filter, the platelet filter and/or the second filter comprises a plurality of filter portions stacked together in a stacking direction and being configured for, seen in the stacking direction, providing alternating first flow paths for retentate of the filter and second flow paths for filtrate of the filter.

10. The blood The blood filtering apparatus according to claim 1, comprising an inlet for the blood to be filtered, connected to plural filter layers and/or comprising a first outlet for retentate of the cell filter optionally connected to plural filter layers and a second outlet for filtrate of the cell filter possibly optionally plural filter layers.

11. A method, of recovering blood components from blood, comprising separating an amount of blood comprising red blood cells and platelets into a red blood cell rich fraction and a red blood cell poor fraction,
wherein the red blood cell rich fraction has a haematocrit of at least 30%; and/or
the red blood cell rich fraction comprises at least 60% of the red blood cells of the ; amount of blood and
wherein the red blood cell rich fraction comprises about 40-450 thousand platelets per microliter and/or
the red blood cell rich fraction comprises at least 25% of the platelets of the amount of blood.

12. The of recovering blood components from blood_{;} according to claim 11, further comprising filtering a portion of the blood using a blood filtering apparatus according to claim 1 and collecting at least one of the retentate and the filtrate, wherein the retentate is a red blood cell rich fraction and the filtrate is a red blood cell poor fraction.

13. The method according to claim 12, further comprising filtering a portion of the filtrate of the cell filter and providing a platelet rich fraction.

14. The method according claim 11, further comprising obtaining blood from a donor that is recovering or has recovered from an affliction for obtaining in the filtrate high levels of antibodies.

15. A blood product for transfusion into a human recipient, wherein the blood product has a haematocrit of at least 30% and comprises about 40-300 thousand platelets per microliter; and wherein the blood product consists essentially of blood components of a single donor individual.

16. The apparatus of claim 1, wherein the pore size is in a range of 2.3-2.4 microns.

17. The apparatus of claim 3, wherein the second filter has a pore size in a range of about 0.3-0.4 microns.

18. The apparatus of claim 4, wherein the polymer layer is a particular a polymer surface layer that is a polymer membrane comprising a polymer material selected from the group consisting of polyester polyurethane, polyethylene terephthalate, polyethylene furanoate, poly(propylene furan-2,5-dicarboxylate).

19. The method of claim 11, wherein the haematocrit is in a range of 30%-60%;
the red blood cell rich fraction comprises at least 75% of the red blood cells of the amount of blood;
the red blood cell rich fraction comprises about 70-400 thousand of the platelets per microliter; and
the red blood cell rich fraction comprises at least 50% of the platelets of the amount of blood.

20. The blood product of claim 15, where the haematocrit is in a range of 30-60% and the blood product contains about 70-150 thousand of the platelets per microliter.
